(19) United States
(12) Patent Application Publication
(10) Pub. No.: US 2024/0075249 A1
(43) Pub. Date: Mar. 7, 2024

(54) Instrument Delivery Device with Single-Handed Advancement Capability

(71) Applicant: Becton, Dickinson and Company, Franklin Lakes, NJ (US)

(72) Inventors: Jonathan Karl Burkholz, Salt Lake City, UT (US); Curtis H. Blanchard, Herriman, UT (US); Weston F. Harding, Lehi, UT (US); S. Ray Isaacson, Layton, UT (US)

(21) Appl. No.: 17/900,995

(22) Filed: Sep. 1, 2022

(51) Int. Cl. A61M 25/01

(52) U.S. Cl. CPC A61M 25/0113

(57) ABSTRACT

Provided herein is an instrument delivery device for advancing an instrument into a vascular access device. The instrument delivery device includes an outer housing, an introducer portion positioned at the distal end of the outer housing and configured to mate with the vascular access device, and an inner housing having a distal end positioned within an inner volume of the outer housing and engaged with a proximal end of the instrument, with the inner housing moveable relative to the outer housing. A wheel member is positioned on the outer housing and rotatable relative thereto and is operably coupled to the inner housing to move the inner housing distally within the outer housing when the wheel member is rotated in a first direction, thereby also moving the instrument distally from to a position where the distal end of the instrument is disposed beyond the distal end of the outer housing.

### BACKGROUND OF THE INVENTION

### Field of the Invention

The present disclosure relates generally to instrument delivery devices for use with intravenous (IV) catheters and, more specifically, to instrument delivery devices with features for advancing an instrument into the vasculature.

### Description of Related Art

Vascular access devices (VADs) are used in the medical field to access peripheral vasculature of a patient for purposes of infusion therapy and/or blood withdrawal. Common types of VADs include over-the-needle peripheral intravenous catheters (PIVCs), peripherally inserted central catheters (PICCs), central venous catheters (CVCs), and midline catheters. The VAD may be indwelling for short term (days), moderate term (weeks), or long term (months to years).

Instrument delivery devices are often used with indwelling intravenous (IV) catheters to deliver an instrument such as a fluid path tubing, guidewire, obturator, wire, electrical wiring, probe, or sensor(s) into the IV catheter, with the instrument delivery device advancing the instrument beyond the tip of the indwelling catheter. Some instrument delivery devices use a nested housing arrangement (i.e., nested outer and inner housings) to advance the instrument through to a position proximal or distal to the tip of the indwelling catheter. The outer housing of the device is positioned distally, at a location adjacent the VAD, with the inner housing extending out proximally from the outer housing. The inner housing may include gripping features thereon that allow an operator to grasp and push the inner housing into the outer housing, thereby also advancing the instrument distally and into the indwelling catheter. One disadvantage of this configuration of the instrument delivery device, and of the method used to advance the inner housing into the outer housing, is that it requires the operator to use two hands to actuate the instrument delivery device. That is, one hand of the operator is positioned distally at the end of the outer housing adjacent the VAD, to steady the instrument delivery device, and the other hand is positioned proximally at the end of the inner housing, to grip and push the inner housing into the outer housing. The hands of the operator are thus positioned a large distance from each other and require significant relative motion to each other in order to operate the device, with neither hand available to stabilize the VAD and indwelling catheter through which the instrument is advancing.

Accordingly, a need exists in the art for instrument delivery devices that allow for ergonomic, one-handed advancement options that enable the advancement hand to stay in a reference position without requiring significant motion axially following the motion of the inner housing.

### SUMMARY OF THE INVENTION

Provided herein is an instrument delivery device for advancing an instrument into a vascular access device. The instrument delivery device includes an outer housing defining an inner volume and having a proximal end and a distal end, an introducer portion positioned at the distal end of the outer housing and configured to mate with an access connector of the vascular access device, and an inner housing having a proximal end and a distal end, with the distal end positioned within the inner volume and engaged with a proximal end of the instrument, and with the inner housing configured to move relative to the outer housing. The instrument delivery device also includes a wheel member positioned on the outer housing and rotatable relative to the outer housing, wherein the wheel member is operably coupled to the inner housing to move the inner housing distally within the outer housing when the wheel member is rotated. The distal movement of the inner housing moves the instrument from a first position, in which a distal end of the instrument is disposed within the outer housing, to a second position, in which the distal end of the instrument is disposed beyond the distal end of the outer housing.

In some embodiments, the wheel member is mounted on the outer housing adjacent the distal end of the outer housing.

In some embodiments, a grip feature is formed on the outer housing, at the distal end of the outer housing adjacent to the wheel member.

In some embodiments, the wheel member includes an advancement wheel engageable by the user to cause rotation thereof and a spool operably coupled with the advancement wheel such that the spool rotates responsive to rotation of the advancement wheel.

In some embodiments, a tether cord is provided having a first end coupled to the distal end of the inner housing and a second end engaged with the spool.

In some embodiments, rotation of the wheel member in a first direction causes the tether cord to wrap around the spool and pull the inner housing toward the distal end of the outer housing, thereby moving the instrument from the first position to the second position.

In some embodiments, the tether cord is a semi-rigid cord and rotation of the wheel member in a second direction causes the semi-rigid cord to unwrap from the spool and push the inner housing back toward the proximal end of the outer housing, thereby moving the instrument from the second position to the first position.

In some embodiments, the spool is mounted adjacent the advancement member and includes a gear mounted thereto, with each of the advancement member and the gear including teeth thereon that mate together such that rotation of the advancement member causes a rotation of the gear that is transferred to the spool.

In some embodiments, the advancement member and the gear have different diameters, so as to provide a mechanical advantage of force or displacement when pulling the tether cord as the advancement member is rotated.

In some embodiments, the instrument runs forward from the proximal end to the distal end of the outer housing, and wherein one of the instrument and the tether cord is looped around the spool one or more times to effectively couple the instrument or the tether cord to the wheel member.

In some embodiments, rotation of the wheel member in a first direction causes the instrument and the inner housing to advance toward the distal end of the outer housing, thereby moving the instrument from the first position to the second position, and rotation of the wheel member in a second direction causes the instrument and the inner housing to retract toward the proximal end of the outer housing, thereby moving the instrument from the second position to the first position.

In some embodiments, during rotation of the wheel member in the first direction and the second direction, a first portion of the instrument is pulled by the wheel member and a second portion of the instrument is pushed by the wheel member.

In some embodiments, the wheel member is mounted on the outer housing adjacent the proximal end of the outer housing, with a bottom side of the wheel member engaging an upper surface of the inner housing, and rotation of the wheel member in a second direction, toward the proximal end of the outer housing, causes the bottom side of the wheel member to urge the inner housing toward the distal end of the outer housing, thereby moving the instrument from the first position to the second position.

In some embodiments, the wheel member is a toothed wheel and the top surface of the inner housing includes a grooved track, with the toothed wheel engaging the grooved track in a rack and pinion type engagement as the toothed wheel is rotated in the second direction, to urge the inner housing toward the distal end of the outer housing.

In some embodiments, one or more support wheels are positioned in the outer housing, within the inner volume, and adjacent a bottom surface of the inner housing, opposite the wheel member, with the wheel member and the one or more support wheels pressing together on the inner housing to provide a friction engagement between the wheel member and the top surface of the inner housing, with the friction engagement between the wheel member and the top surface of the inner housing urging the inner housing toward the distal end of the outer housing as the wheel member is rotated in the second direction.

In some embodiments, the wheel member is a compliant advancement wheel configured to provide the friction engagement between the wheel member and the top surface of the inner housing.

In some embodiments, the instrument is one or more of a catheter, guidewire, obturator, wire, electrical wiring, probe, light pipe, and sensor.

In some embodiments, the instrument is a catheter, and the inner housing is in fluid communication with the catheter, such that fluid flowing proximally from the catheter is received within the inner housing or fluid flowing distally from the inner housing is received in the catheter.

In some embodiments, one or more supports arranged within the outer housing and configured to limit buckling of the instrument as it is advanced through the outer housing.

Also provided herein is a system, including a catheter assembly having a catheter adapter with a lumen extending between a distal end and a proximal end of the catheter adapter and a side port arranged between the distal end and the proximal end, the side port in fluid communication with the lumen. The catheter assembly also includes a catheter secured to the distal end of the catheter adapter and extending distally from the catheter adapter, and a fluid conduit having a proximal end coupled to the side port and a distal end coupled to a needless access connector. An instrument delivery device advances an instrument through the needless access connector, the fluid conduit, and the catheter adapter, and into the catheter.

### BRIEF DESCRIPTION OF THE DRAWINGS

FIG. 1A shows a side view of a catheter assembly and an associated instrument delivery device useable therewith, with the instrument delivery device in a first configuration, according to a non-limiting embodiment described herein;

FIG. 1B shows a side view of the catheter assembly and associated instrument delivery device of FIG. 1A, with the instrument delivery device in a second configuration;

FIG. 2 shows a perspective view of the instrument delivery device of FIGS. 1A and 1B;

FIG. 3 shows a partial cross-section view of the instrument delivery device of FIG. 2, taken along line a-a, illustrating a wheel member, according to an embodiment described herein;

FIG. 4 shows an isolated end view of the wheel member of FIG. 3;

FIG. 5 shows a partial cross-section view of the instrument delivery device of FIG. 2, taken along line a-a, illustrating a wheel member, according to another embodiment described herein;

FIG. 6 shows an isolated end view of the wheel member of FIG. 3;

FIG. 7 shows a perspective view of an instrument delivery device according, according to a non-limiting embodiment described herein;

FIG. 8 shows a partial cross-section view of the instrument delivery device of FIG. 7, taken along line b-b, illustrating a wheel member, according to an embodiment described herein;

FIG. 9 shows an isolated end view of the wheel member of FIG. 8;

FIG. 10 shows a partial cross-section view of the instrument delivery device of FIG. 7, taken along line b-b, illustrating a wheel member, according to another embodiment described herein;

FIG. 11 shows an isolated end view of the wheel member of FIG. 10;

FIG. 12A shows a side view of a catheter assembly and an associated instrument delivery device useable therewith, with the instrument delivery device in a first configuration, according to a non-limiting embodiment described herein;

FIG. 12B shows a side view of the catheter assembly and associated instrument delivery device of FIG. 12A, with the instrument delivery device in a second configuration;

FIG. 13 shows a perspective view of the instrument delivery device of FIGS. 12A and 12B;

FIG. 14 shows a partial cross-section view of the instrument delivery device of FIG. 13, taken along line c-c, according to an embodiment described herein;

FIG. 15 shows a partial cross-section view of the instrument delivery device of FIG. 13, taken along line c-c, according to another embodiment described herein.

FIG. 16 shows a partial cross-section view of an instrument delivery device, illustrating a wheel member, according to another embodiment described herein;

FIG. 17 shows a partial cross-section view of an instrument delivery device, illustrating a wheel member, according to another embodiment described herein; and

FIG. 18 shows an isolated end view of the wheel member of FIG. 17.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, equivalents, variations, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

As used in this specification, the words “proximal” and “distal” refer to the direction closer to and away from, respectively, a user who would place the device into contact with a patient. Thus, for example, the end of a device first touching the body of the patient would be the distal end, while the opposite end of the device being manipulated by the user would be the proximal end of the device.

The terms “first”, “second”, and the like are not intended to refer to any particular order or chronology, but refer to different conditions, properties, or elements.

As used herein, “at least one of” is synonymous with “one or more of.” For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

It should be understood that any numerical range recited herein is intended to include all values and sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value equal to or less than 10.

Provided herein are devices and systems for delivering instruments through indwelling catheters, such as peripheral intravenous catheters (PIVCs). While certain devices (e.g., blood draw devices) are discussed below in terms of devices that may be used with PIVCs, and exemplified in the attached drawings, those of skill will appreciate that any number of different devices for introducing an instrument, including instruments ranging from tubes, probes, sensors (e.g., pressure sensors, pH sensors, lactate sensors, glucose sensors, and the like), wiring, fiber optics, guidewires, etc., may be used within the scope of the present disclosure.

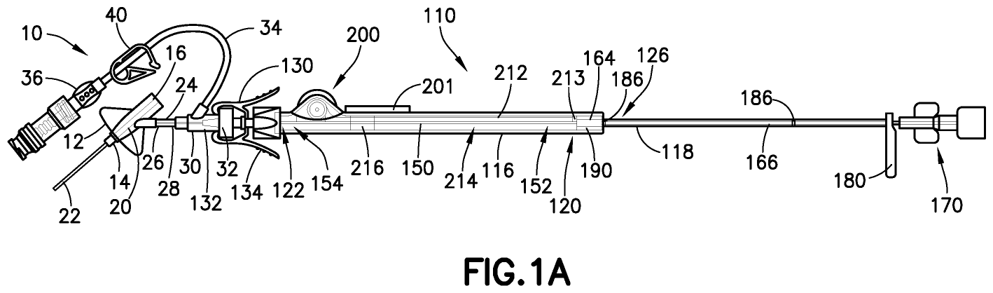

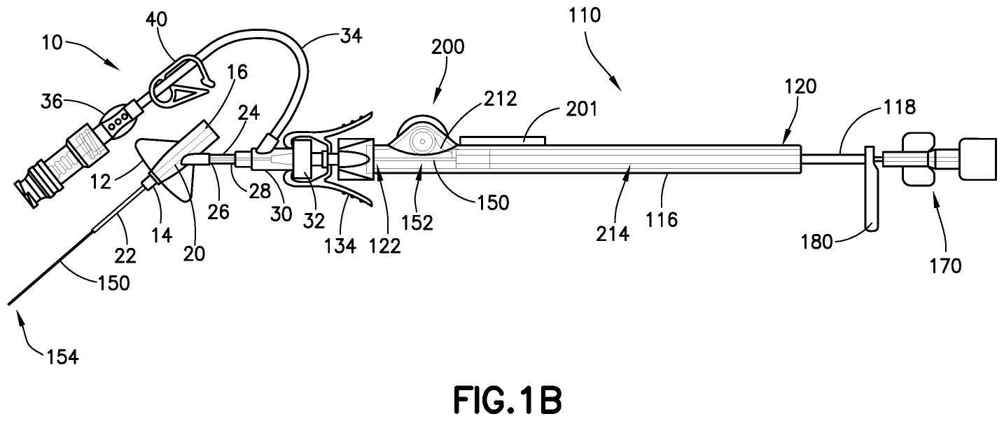

Referring now to FIGS. 1A and 1B, shown is a non-limiting embodiment of a system including a catheter assembly 10 and instrument delivery device 110. Suitable catheter assemblies for use with instrument delivery devices described herein are commercially available, for example from Becton, Dickinson and Company under the trade name Nexiva. Catheter assembly 10 may include a catheter adapter 12, which may include a distal end 14 and a proximal end 16. In some embodiments, the catheter adapter 12 may include one or more additional ports 18. The port 18 may be disposed between the distal end 14 and the proximal end 16, and more than one port 18 may be disposed between the distal end 14 and the proximal end 16. The port 18 may instead be disposed at proximal end 16. In some embodiments, the first catheter adapter 12 may include a first lumen 20 extending through the distal end 14 and the proximal end 16. First lumen 20 may be sealed at proximal end 16 of catheter adapter 12.

In some non-limiting embodiments or aspects, the catheter assembly 10 may include a catheter 22 extending from the distal end 14. The first catheter 22 may include a peripheral intravenous catheter, a midline catheter, or a peripherally-inserted central catheter. Catheter 22 may be formed of any suitable material and may be of any useful length, as known to those of skill in the art. In some non-limiting embodiments or aspects, the catheter assembly 10 may include a first fluid conduit 24 extending from the port 18. First fluid conduit 24 may be formed of any suitable material known to those of skill in the art, and may have a distal end 26 and a proximal end 28, and first fluid conduit 24 may be coupled, at distal end 26 thereof, to port 18. In some non-limiting embodiments or aspects, a connector 30 may be coupled to a proximal end 28 of first fluid conduit 24. Connector 30 may be a t-connector (e.g., one side port arranged at a 90 degree angle relative to a longitudinal axis of connector 30), a y-connector (e.g., one side port arranged at an angle of 15-165 degrees relative to a longitudinal axis of connector 30), or any other type of connector known in the art, and may include a second lumen therethrough, having any number of branches suitable for the type of connector.

In some non-limiting embodiments or aspects, catheter assembly 10 may include an extension set (integrated into or removably coupleable to catheter adapter 12, connector 30, and/or needleless access connector 32) including a second fluid conduit, such as second fluid conduit 34. Extension sets are known to those of skill in the art and are commercially available from, for example, Becton, Dickinson and Company. In some non-limiting embodiments or aspects, second fluid conduit 34 may include a luer connection 36 at an end thereof. The extension set may also include a clamp 40, to allow for occlusion of second fluid conduit 34. Clamp 40 and second fluid conduit 34 may be formed of any suitable materials known to those of skill in the art. In non-limiting embodiments, second lumen (e.g., within connector 30) has an inner diameter that is substantially equivalent to an inner diameter of first fluid conduit 24 and/or second fluid conduit 34.

Catheter assembly 10 may include a needleless access connector 32 and/or a second fluid conduit 34. Suitable needleless access connectors 32 can include any split-septum connector and/or those with direct fluid path access. Needleless access connectors 32 are known to those of skill in the art and are commercially available from, for example, Becton, Dickinson and Company under the trade names Q-SYTE and SMARTSITE. While the non-limiting embodiments of FIGS. 1A and 1B show needleless access connectors arranged at connector 30, those of skill in the art will appreciate that suitable needleless access connectors may also be arranged at luer 36. In non-limiting embodiments, needleless access connector 32 includes a septum (not shown), such as a slit-type, self-healing septum. As will be described below, instrument delivery device 110 may be reversibly coupleable to the needleless access connector 32, and one or more portions of the instrument delivery device may pierce the septum and access the patient's vasculature through catheter 22.

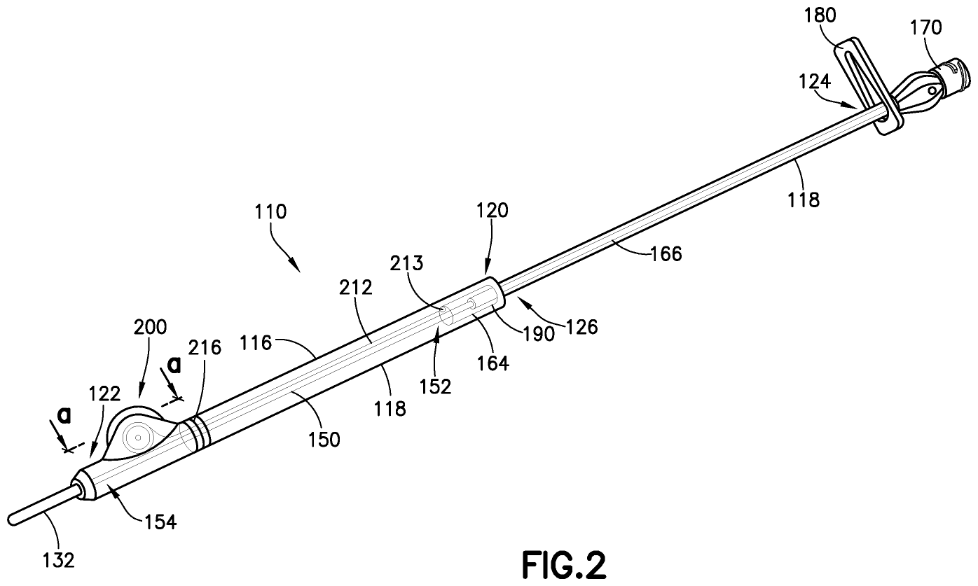

With continuing reference to FIGS. 1A and 1B and now also to FIG. 2, instrument delivery device 110 includes an outer housing 116 having a proximal end 120 and a distal end 122, and an inner housing 118 slideably received within outer housing 116. In non-limiting embodiments, inner housing 118 and outer housing 116 are in a telescoping relationship, such that inner housing 118 may be slideably received entirely, or almost entirely, within outer housing 116. Inner housing 118 also includes a proximal end 124 and a distal end 126 and, in non-limiting embodiments, inner housing may have a variable diameter along its length. As one example, the distal end 126 of inner housing 118 has a larger diameter than other portions of inner housing 118, to provide more local stiffness or a sliding feature(s) to limit friction or binding. As another example, the distal end 126 of inner housing 118 has a smaller diameter than other portions of inner housing 118, to position to keep the inner housing 118 in position at a blood draw forward condition, so that a hand of the operator is freed up to manipulate additional components (e.g., a vacutainer tube). Instrument delivery device 110 further includes an instrument, exemplified in the drawings as a catheter or fluid conduit 150 (but understood to alternatively be a probe, guidewire, or other instrument) having a proximal end 152 and a distal end 154, but, as described previously and as will be appreciated by those of skill in the art, may be any medical instrument that can be delivered through catheter assembly 10 to a patient's vasculature. Fluid conduit 150 is received within outer housing 116, and may be advanced and/or retracted relative to outer housing 116 by displacement of the inner housing 118 relative to the outer housing 116. In non-limiting embodiments, fluid conduit 150 may be advanced from a first positon as shown in FIG. 1A, in which distal end 154 of fluid conduit 150 is within instrument delivery device 110, for example within outer housing 116 and/or a lock 130 (that secures instrument delivery device 110 to needleless access connector 32, for example), to a second position as shown in FIG. 1B, in which a distal end 154 of fluid conduit 150 is positioned distally of lock 130 and, in embodiments in which instrument delivery device 110 is coupled to catheter assembly 10, optionally distally of catheter 22. While lock 130 is exemplified as a blunt cannula 132 and arms 134, those of skill will appreciate that any type of suitable connection may be used to secure instrument delivery device 110 to an indwelling catheter, such as catheter assembly 10, including luer connections, clips, blunt plastic cannulae, blunt metal cannulae, hybrid luers (e.g., with a cannula) friction fits, and the like.

Instruments useful with the instrument delivery device 110 described herein may be formed of any useful material. In non-limiting embodiments, instrument is a fluid conduit, which is formed of a polymer, such a polyimide-containing material. In addition, inner housing 118 may be formed of any suitable material, including materials that provide the inner housing with a flexible, rigid, or semi-rigid structure. In non-limiting embodiments, inner housing 118 is formed of a material that provides resistance to buckling.

As can be appreciated, FIG. 1A shows instrument delivery device in a first state, where instrument (here, fluid conduit 150) in a first position, received within outer housing 116, and inner housing 118 is in a first position, extending proximally from outer housing. In non-limiting embodiments, inner housing 118 is coupled to or otherwise interacts with fluid conduit 150, such that as inner housing 118 is advanced distally to a second position, as shown in FIG. 1B, fluid conduit 150 is moved to a second position, where a distal end 154 of fluid conduit 150 extends beyond outer housing 116, lock 130 (if present), and/or catheter 22.

As described above, a distal end of inner housing 118 may be of a larger diameter than other portions of inner housing 118, such that, as inner housing 118 is retracted, one or more features on outer housing 116 may interact with the enlarged portion of inner housing 118 to prevent pulling inner housing 118 completely out of outer housing 116. Enlarged distal portion of inner housing 118 may include vents for allowing air to pass therethrough, reducing force needed to advance/retract inner housing 118, and, as described below, lubricant 190 may be applied to an enlarged portion of inner housing 118 to reduce friction between inner housing 118 and outer housing 116.

Inner housing 118 may include, at the proximal end 124 thereof, a connector 170, to allow for various medical devices to be attached to inner housing 118, for example to provide an instrument that is to be advanced into the patient's vasculature, to inject a composition into the vasculature, and/or to receive fluid withdrawn from the vasculature. Suitable connectors 170 include luer connectors, luer lock access devices, needless access connectors, and the like known to those of skill in the art.

In non-limiting embodiments inner housing 118 may include one or more indicia 186 arranged on an outer surface thereof. Suitable indicia may be visual and/or tactile, and may be provided to, for example, indicate instrument length, instrument positioning relative to indwelling catheter. In non-limiting embodiments, one or more indicia 186 may be provided on inner housing 118, and outer housing 116 may be formed of a material that is at least partially transparent, to allow for visualization of indicia 186 throughout the transition from a first positon of inner housing 118 to a second position of outer housing 118. In non-limiting embodiments, indicia 186 in the form of a tactile stop may be included at one or more locations along inner housing 118, to provide a user with a tactile indication of certain thresholds. For example, a tactile stop may indicate that instrument (e.g., fluid conduit 150) has nearly reached its full extension and/or may indicate that the instrument can be extended no further (for example, as shown in FIG. 1B).

In non-limiting embodiments a lubricant 190 may be applied at one or more locations on or within one or more components of instrument delivery device 110. For example, a lubricant 190 may be applied to an outer surface of a distal portion (e.g., blunted cannula 132) of outer housing 116 of instrument delivery device 110 that is inserted into an indwelling catheter, such as catheter assembly 10, to decrease the force necessary to couple the devices together. A lubricant 190 may be applied at one or more locations within outer housing 116, for example on one or more outer surfaces of inner housing 118 and/or on one or more outer surfaces of instrument (e.g., fluid conduit 150).

In non-limiting embodiments, instrument delivery device 110 includes one or more supports 216 arranged within outer housing 116, to limit and/or prevent buckling of inner housing 118 and/or instrument (e.g., fluid conduit 150) as inner housing 118 and/or the instrument are advanced distally through outer housing 116. Supports 216 may include narrowed portions of outer housing 116, one or more washers arranged about inner housing 118 and/or instrument, and/or the like, for example to reduce the effective buckling length and/or buckling mode shape of inner housing 118 and/or instrument. Suitable supports are also described in U.S. Provisional Patent Application No. 63/273,226, filed Oct. 29, 2021, the contents of which are incorporated herein by reference in their entirety.

In non-limiting embodiments where instrument is a fluid conduit 150, fluid (e.g., blood) may be transferred into or from the patient's vasculature in which a catheter, such as catheter 22, may be indwelling. As shown in FIGS. 1A and 1B and FIG. 2, fluid conduit 150 may be joined at fitting 164 to inner housing 118. According to non-limiting embodiments, fluid conduit 150 may be joined at fitting 164 to a separate fluid tube 166 that passes through inner housing 118 and is coupled to connector 170. In other non-limiting embodiments, fluid conduit 150 may be joined at fitting 164 to inner housing 118, with inner housing 118 itself forming a fluid conduit in fluid communication with connector 170. In either of the above described embodiments, a clamp 180 may be provided at the proximal end 124 of inner housing 118 that is suitable for occluding fluid flow through inner housing 118 and/or fluid conduit 166. Clamp 180 may be a slide clamp or a pinch clamp, according to embodiments.

According to embodiments, movement of inner housing 118 and fluid conduit 150 relative to outer housing 116, including distal movement and optionally proximal movement thereof, is effected by way of a wheel member 200 that is provided on the outer housing 116. In operation of the instrument delivery device 110, an operator actuates (e.g., rotates) the wheel member 200, which in turn causes a linear movement of the inner housing 118 and fluid conduit 150, such as advancement or retraction of the inner housing 118 and fluid conduit 150. By providing the wheel member 200 on the outer housing 116, the operator may use a single hand to both steady the instrument delivery device 110 (relative to the catheter assembly 10/catheter adapter 12) and operate the wheel member 200 to translate the inner housing 118 and fluid conduit 150 between the first position and second position described above. Accordingly, the wheel member 200 allows for ergonomic, one-handed advancement options that allow the advancement hand to stay in a reference position without requiring significant motion axially following the motion of the inner housing 118.

In one embodiment, a grip 201 is formed on an outer surface of the outer housing 116 adjacent the wheel member 200. While a thumb or other finger of the operator engages the wheel member 200, a remainder of the operator's hand may grasp onto the grip 201, such that the instrument delivery device 110 may be firmly retained by the operator.

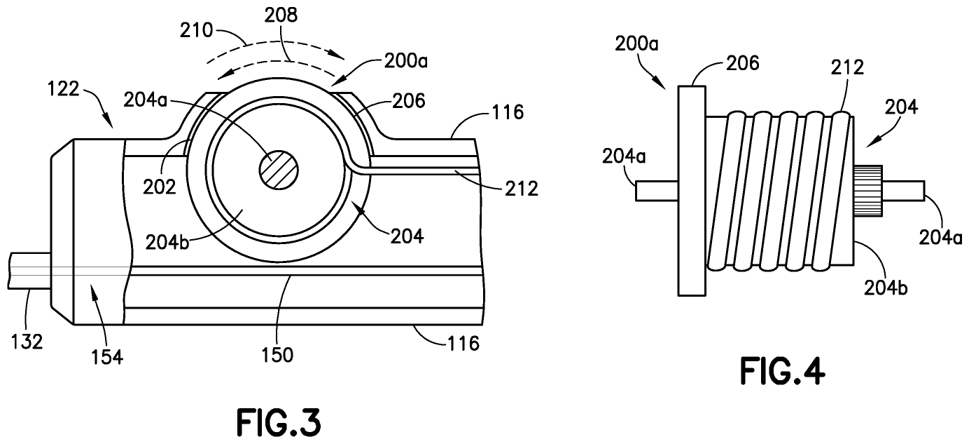

FIGS. 3 and 4 illustrate an example embodiment of a wheel member 200—wheel member 200a—that may be included in instrument delivery device 110. The wheel member 200a is positioned on outer housing 116 at the distal end 122 thereof. Outer housing 116 includes a compartment 202 formed therein configured to house the wheel member 200a. Wheel member 200a includes a spool 204 and an advancement wheel 206 formed or coupled together to form a single component. The spool 204 includes an axle 204a that maintains spool 204 within compartment 202 and allows the spool 204 to rotate. The spool 204 also includes a spool drum 204b around which a tether cord 212 is wound, as explained in more detail below. The advancement wheel 206 that is directly coupled to or formed with spool 204 extends upwardly from compartment 202, so as to be engageable by an operator. An operator can directly rotate advancement wheel 206 by applying a force thereto, with the advancement wheel 206 rotatable in a first direction 208 and a second direction 210.

Actuation of the wheel member 200a causes a corresponding linear movement of the inner housing 118 and fluid conduit 150 by way of a tether cord 212 that couples the inner housing 118 to wheel member 200a. A first end of the tether cord 212 is connected to the distal end 126 of inner housing, such as at an anchor point 213, and a second end of the tether cord 212 is wound about the spool drum. The tether cord 212 extends within the volume 214 defined by outer housing 116 and, in one embodiment, may be positioned above the fluid conduit 150 so as to avoid contact therewith. Supports 216 and/or other components contained with outer housing 116 (e.g., seals) may be configured to accommodate routing of the tether cord 212.

According to non-limiting embodiments, tether cord 212 may be formed of any of a number of suitable materials capable of withstanding forces applied thereto via operation of the associated wheel member 200a. In some embodiments, the tether cord 212 may be configured to only withstand tension forces applied thereto, with tension forces applied responsive to rotation of advancement wheel 206 in the first direction 208. In such embodiments, tether cord 212 may be formed of a flexible polymeric material, for example. In other embodiments, the tether cord 212 may be configured to withstand both tension and compression/pushing forces applied thereto, with tension forces applied responsive to rotation of advancement wheel 206 in the first direction 208 and compression/pushing forces applied responsive to rotation of advancement wheel 206 in the second direction 210. In such embodiments, tether cord 212 may be formed of a semi-rigid metallic or polymeric material (e.g., a metal wire or guidewire) able to withstand both tension forces and compression/pushing forces

In operation of the instrument delivery device 110, rotation of the wheel member 200a (i.e., rotation of advancement wheel 206, and a corresponding rotation of spool 204) in the first direction 208 (i.e., toward the distal end 122 of the outer housing 116) causes the tether cord 212 to wrap around the spool drum 204b, thereby applying a pulling force to the inner housing 118. Responsive to application of this pulling force, the inner housing 118 is caused to move/slide toward the distal end 122 of the outer housing 116, and this, in turn, causes the fluid conduit 150 to advance distally, such that the distal end 154 of the fluid conduit 150 may be extended out from outer housing 116 and blunted cannula 132 and into catheter assembly 10.

In some embodiments, the inner housing 118 and fluid conduit 150 may be retracted back toward the proximal end 120 of outer housing 116 by an operator manually pulling the proximal end 124 of the inner housing 118 back out of the outer housing 116. In other embodiments, rotation of the wheel member 200a in the second direction 210 (i.e., toward the proximal end 120 of the outer housing 116), causes the tether cord 212 to unwrap from the spool drum 204b, thereby applying a pushing force to the inner housing 118. Responsive to application of this pushing force, the inner housing 118 is caused to move/slide toward the proximal end 120 of the outer housing 116, and this, in turn, causes the fluid conduit 150 to retract proximally, such that the distal end 154 of the fluid conduit 150 may be retracted back into outer housing 116.

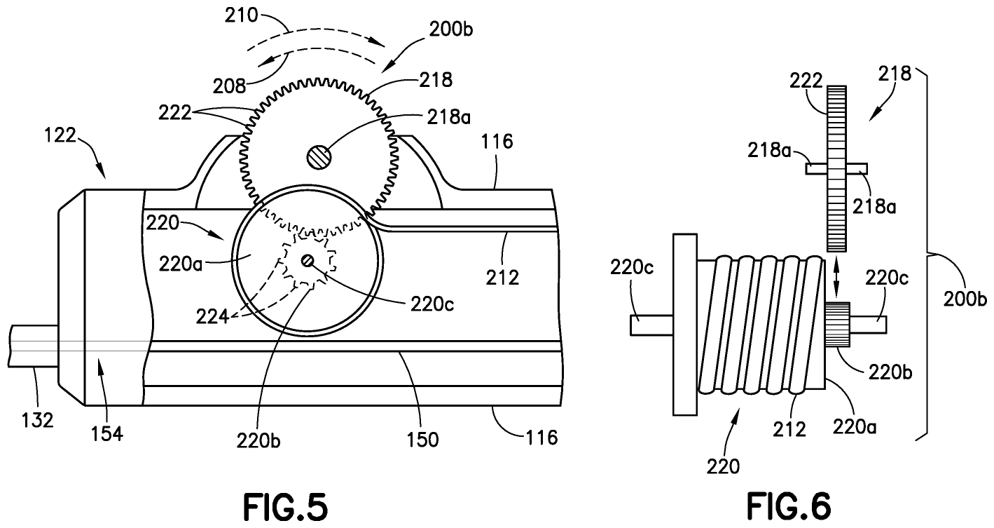

Referring now to FIGS. 5 and 6, another example embodiment of a wheel member 200—wheel member 200b—that may be included in instrument delivery device 110 is shown. Similar to wheel member 200a, the wheel member 200b is positioned on outer housing 116 at the distal end 122 thereof and within a compartment 202 of the outer housing 116. The wheel member 200b includes an advancement wheel 218 and a spool 220, both of which are configured to rotate within compartment 202. Advancement wheel 218 is positioned to extend partially out from compartment 202 to thereby enable an operator to use his or her thumb or finger to rotate the advancement wheel 218. The advancement wheel 218 includes teeth 222 thereon and may therefore function as a gear, as explained in more detail below. The spool 220 is positioned adjacent to advancement wheel 218 and horizontally offset therefrom. The spool 220 includes a spool drum 220a around which tether cord 212 may be wound and a gear 220b having teeth 224 that interface with the teeth 222 on advancement wheel 218, so that spool 220 is rotated when advancement wheel 218 is rotated. In the depicted embodiment, teeth 222 are formed along the outermost edge of advancement wheel 218. In other embodiments, however, teeth 222 may be formed along a portion of advancement wheel 218 that is inset relative to the outermost edge.

As shown in FIGS. 5 and 6, spool 220 and advancement wheel 218 include axles 220c and 218a, respectively, by which these components are positioned within compartment 202 and around which these components rotate. When spool 220 is rotated, the rotation may cause the tether cord 212 to be wound on or unwound from spool drum 220a depending on the direction in which advancement wheel 218 is rotated (i.e., tether cord 212 is wound on spool drum 220a when advancement wheel 218 is rotated in second direction 210, and unwound from spool drum 220a when advancement wheel 218 is rotated in first direction 208), thereby causing a corresponding distal or proximal displacement of the inner housing 118 and fluid conduit 150 relative to outer housing 116. That is, rotation of the advancement wheel 218 in the second direction 210 causes the tether cord 212 to wrap around the spool drum 220a, thereby applying a pulling force to the inner housing 118 and causing the inner housing 118 to move/slide toward the distal end 122 of the outer housing 116 and fluid conduit 150 to also advance distally. Additionally, in some embodiments, rotation of the advancement wheel 218 in the first direction 208 causes the tether cord 212 to unwrap from the spool drum 220a, thereby applying a pushing force to the inner housing 118 and causing the inner housing 118 to move/slide toward the proximal end 120 of the outer housing 116 and fluid conduit 150 to retract proximally.

According to the depicted embodiment, the gear formed by advancement wheel 218 has a larger diameter than gear 220b to thereby cause tether cord 212—and the inner housing 118 (and fluid conduit 150) coupled thereto—to be advanced or retracted a larger distance relative to the amount of rotation of advancement wheel 218. That is, the larger diameter of advancement wheel 218 provides a displacement-type mechanical advantage in the instrument delivery device 110 when advancing or retracting inner housing 118 and fluid conduit 150. In contrast, in other embodiments, the gear formed by advancement wheel 218 may have an equal or smaller diameter than gear 220b. In such embodiments, inner housing 118 (and fluid conduit 150) may advance or retract a smaller distance relative to the amount of rotation of advancement wheel 218, but such advancement or retraction may be accomplished with a reduced amount of force to advancement wheel 218.

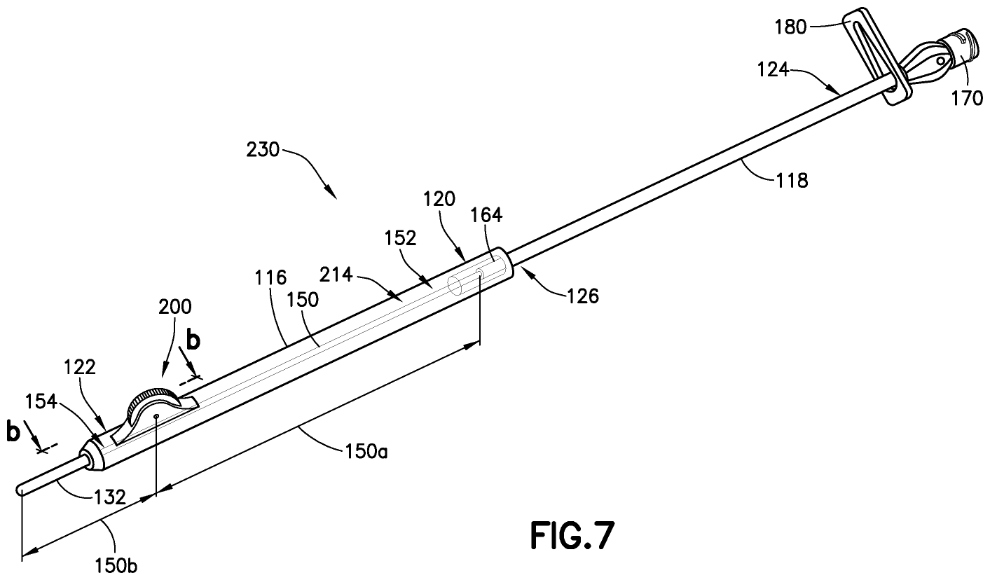

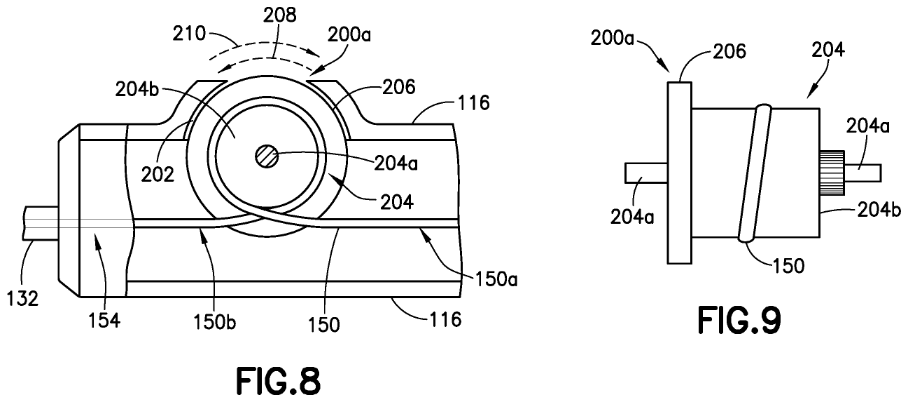

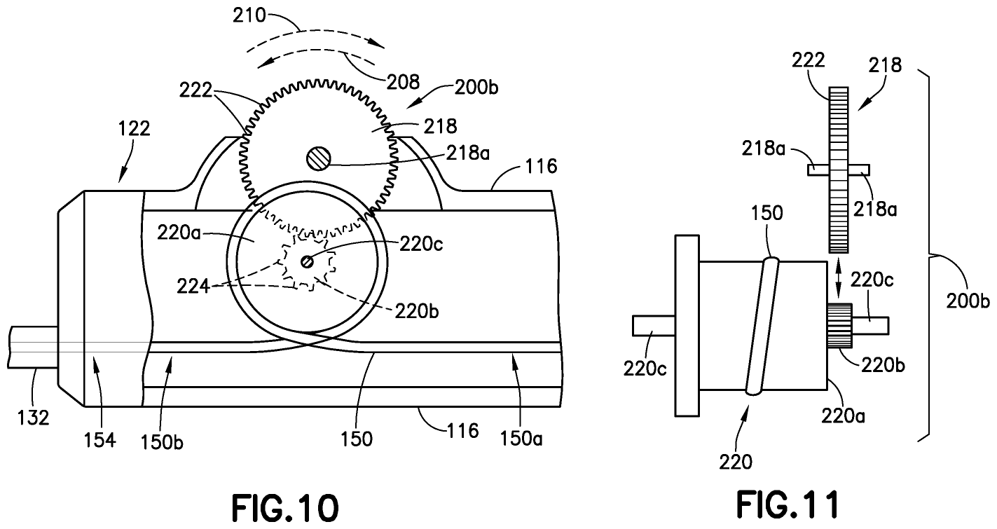

Referring now to FIGS. 7-11, additional embodiments of an instrument delivery device 230 are shown. The structure of instrument delivery device 230 is substantially similar to that of instrument delivery device 110 shown in FIGS. 1-6, and thus like members are labeled identically in instrument delivery device 230. Additionally, the structure of the wheel member 200 included in the instrument delivery device 230—either wheel member 200a (FIGS. 8 and 9) or wheel member 200b (FIGS. 10 and 11)—is identical to that of wheel members 200a, 200b shown in FIGS. 3 and 4 and FIGS. 5 and 6, respectively. As provided in detail here below, the wheel member 200 provided in instrument delivery device 230 interacts with inner housing 118 and fluid conduit 150 of instrument delivery device 230 in a different fashion than the instrument delivery device 110 of FIGS. 1-6, when advancing and retracting inner housing 118 and fluid conduit 150 relative to outer housing 116.

Referring first to FIGS. 8 and 9, wheel member 200a includes a spool 204 and advancement wheel 206 formed or coupled together to form a single component. The spool includes axle 204a that maintains spool 204 within compartment 202 and allows the spool 204 to rotate, as well as spool drum 204b around which fluid conduit 150 is looped, as explained in more detail below. The advancement wheel 206 extends upwardly from compartment 202, such that an operator can directly rotate advancement wheel 206 by applying a force thereto, with the advancement wheel 206 rotatable in a first direction 208 and a second direction 210.

As shown in FIG. 7 and in FIGS. 8 and 9, fluid conduit 150 (or, alternatively, an instrument, such as a probe or guidewire) is looped about the spool drum 204b at a location on the fluid conduit 150 between the proximal end 152 and the distal end 154 thereof, with the fluid conduit 150 therefore operatively connecting or coupling the wheel member 200a to the inner housing 118 (as proximal end 152 of fluid conduit 150 is connected to fitting 164 on inner housing 118). According to embodiments, the fluid conduit 150 is looped about the spool drum 204b one or multiple times (e.g., two, three or more), as required to create traction/friction and a “locking” effect whereby the fluid conduit 150 does not slip relative to the spool 204 when the wheel member 200a is rotated. Accordingly, actuation of the wheel member 200a causes a corresponding linear movement of the fluid conduit 150—and in turn the inner housing 118.

In operation of the instrument delivery device 230, rotation of the wheel member 200a (i.e., rotation of advancement wheel 206 and a corresponding rotation of spool 204) in a first direction 208 (i.e., toward the distal end 122 of the outer housing 116) applies a pulling force to the portion 150a of the fluid conduit 150 extending between the spool drum 204b and inner housing 118, thereby also applying a pulling force to the inner housing 118 that causes the inner housing 118 to move/slide toward the distal end 122 of the outer housing 116. As this portion 150a of fluid conduit 150 is pulled toward the distal end 122 of outer housing 116, the remaining portion 150b of the fluid conduit 150 (between the spool drum 204b and the distal end 154 of fluid conduit 150) is caused to be pushed and advanced distally, such that the distal end 154 of the fluid conduit 150 may be caused to extend out from outer housing 116 and blunted cannula 132 and into catheter assembly 10.

In further operation of the instrument delivery device 230, rotation of the wheel member 200a in the second direction 210 (i.e., toward the proximal end 120 of the outer housing 116) applies a pulling force to the portion 150b of the fluid conduit 150. As this portion 150b of fluid conduit 150 is pulled back and retracted toward the proximal end 122 of outer housing 116, the portion 150a of the fluid conduit 150 (extending between the spool drum 204b and inner housing 118) is caused to be pushed back proximally, thereby also applying a pushing force to the inner housing 118 that causes the inner housing 118 to move/slide back toward the proximal end 120 of the outer housing 116. In other embodiments, the inner housing 118 and fluid conduit 150 may be retracted back toward the proximal end 120 of outer housing 116 by an operator manually pulling the proximal end 124 of the inner housing 118 back out of the outer housing 116.

Referring now to FIGS. 10 and 11, interaction of fluid conduit 150 with wheel member 200b is described according to the illustrated embodiment. As previously described, wheel member 200b includes an advancement wheel 218 and a spool 220, both of which are configured to rotate within compartment 202. Advancement wheel 218 is positioned to extend partially out from compartment 202 and includes teeth 222 thereon and may therefore function as a gear. The spool 220 includes a spool drum 220a around which fluid conduit 150 may be looped one or more times, as explained above, and a gear 220b having teeth 224 that interface with the teeth 222 on advancement wheel 218, so that spool 220 is rotated when advancement wheel 218 is rotated.

Fluid conduit 150 is looped about the spool drum 220a, with the fluid conduit 150 being looped one or multiple times (e.g., two, three or more) about spool drum 220a, as required to create traction/friction and a “locking” effect whereby the fluid conduit 150 does not slip relative to the spool 220 when the wheel member 200b is rotated. Rotation of the wheel member 200b in second direction 210 applies a pulling force to the portion 150a of the fluid conduit 150 extending between the spool drum 220a and inner housing 118, thereby also applying a pulling force to the inner housing 118 that causes the inner housing 118 to move/slide toward the distal end 122 of the outer housing 116. As this portion 150a of fluid conduit 150 is pulled toward the distal end 122 of outer housing 116, the remaining portion 150b of the fluid conduit 150 (between the spool drum 220a and the distal end 154 of fluid conduit 150) is caused to be pushed and advanced distally, such that the distal end 154 of the fluid conduit 150 may be caused to extend out from outer housing 116 and blunted cannula 132 and into catheter assembly 10. Conversely, rotation of the wheel member 200b in the first direction 208 applies a pulling force to the portion 150b of the fluid conduit 150. As this portion 150b of fluid conduit 150 is pulled back and retracted toward the proximal end 122 of outer housing 116, the portion 150a of the fluid conduit 150 (extending between the spool drum 220a and inner housing 118) is caused to be pushed back proximally, thereby also applying a pushing force to the inner housing 118 that causes the inner housing 118 to move/slide back toward the proximal end 120 of the outer housing 116. In other embodiments, the inner housing 118 and fluid conduit 150 may be retracted back toward the proximal end 120 of outer housing 116 by an operator manually pulling the proximal end 124 of the inner housing 118 back out of the outer housing 116.

As previously described, sizing of the advancement wheel 218 as compared to gear 220b (i.e., diameters thereof) can dictate a mechanical advantage provide by the wheel member 200b. With the gear formed by advancement wheel 218 having a larger diameter than gear 220b, the fluid conduit 150 is advanced or retracted a larger distance relative to the amount of rotation of advancement wheel 218. Conversely, with the gear formed by advancement wheel 218 having a smaller diameter than gear 220b, the fluid conduit 150 is advanced or retracted a smaller distance relative to the amount of rotation of advancement wheel 218, but such advancement or retraction may be accomplished with a reduced amount of force to advancement wheel 218.

While FIGS. 7-11 show and describe fluid conduit 150 (or another instrument) looped about spool drum 220a so as to provide for advancement of inner housing 118 and fluid conduit 150, it is recognized that a tether cord 212 (FIGS. 1-6) could instead be looped about spool drum 220a so as to provide for advancement of inner housing 118 and fluid conduit 150. That is, similar to instrument delivery device 110 of FIGS. 1-6, a tether cord 212 could extend between inner housing 118 and wheel member 200a, 200b, but instead of being wound about a spool drum that is part of the wheel member 200a, 200b (i.e., spool drum 220a), the tether cord 212 is only looped about spool drum 220a a sufficient number of times to create traction/friction and a “locking” effect (e.g., one, two or three times, for example) and then continues distally to a separate drum about which the tether cord 212 is then wound. The advancement or retraction of the tether cord 212, via operation of the wheel member 200a, 200b as described above, would then cause a corresponding advancement or retraction of fluid conduit 150 (or alternative instrument) and inner housing 118, with the benefit that looping of tether cord 212 about the spool drum 220a allows the tether cord 212 to better withstand pushing and pulling forces applied thereto to enable advancement and retraction of the fluid conduit 150 and inner housing 118.

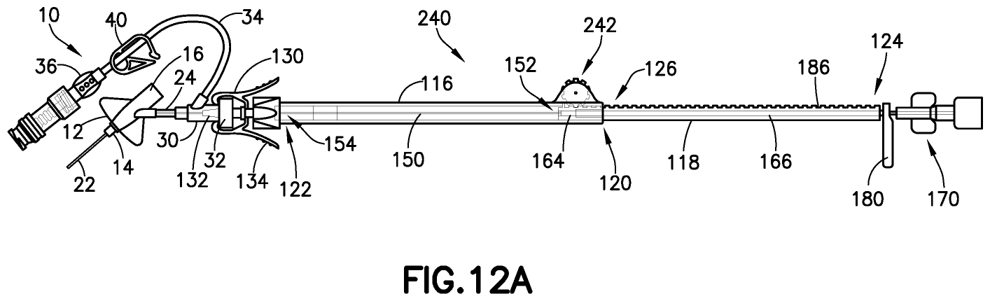

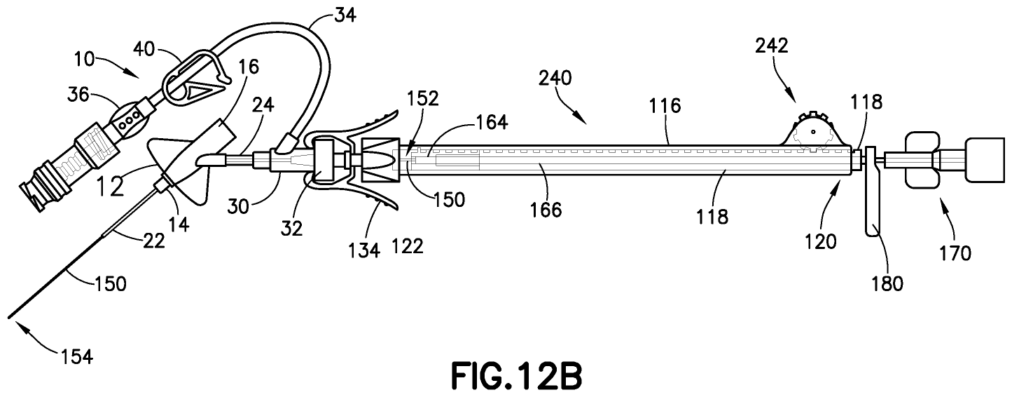

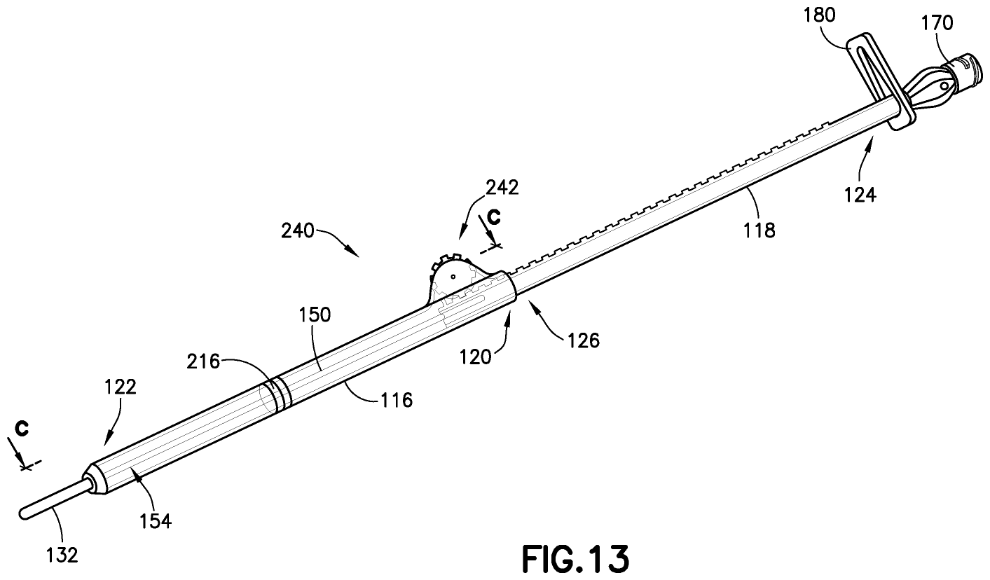

Referring now to FIGS. 12A and 12B and FIG. 13, an instrument delivery device 240 is shown according to another embodiment. Again, components common to the instrument delivery device 240 and to instrument delivery devices 110, 230 are labeled identically.

In instrument delivery device 240, a wheel member 242 is provided for advancing and retracting inner housing 118 and fluid conduit 150. In operation of the instrument delivery device 110, an operator actuates (e.g., rotates) the wheel member 242, which in turn causes a linear movement of the inner housing 118 and fluid conduit 150, such as advancement or retraction of the inner housing 118 and fluid conduit 150. The wheel member 242 is positioned on outer housing 116 at the proximal end 120 thereof. While distinct from the positioning of a wheel member 242 at the distal end 122 of the outer housing 116, as provided in the embodiments of FIGS. 1-11, positioning of wheel member 242 at proximal end 120 on the outer housing 116 still allows for ergonomic, one-handed advancement options for an operator when translating the inner housing 118 and fluid conduit 150 of instrument delivery device 240 between the first position and second position.

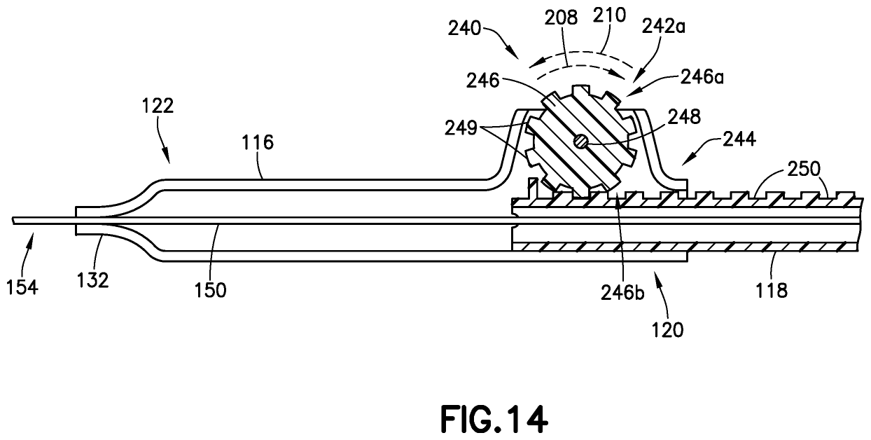

FIG. 14 illustrates an example embodiment of a wheel member 242—i.e., wheel member 242a—that may be included in instrument delivery device 240. The wheel member 242a is positioned on outer housing 116 at the proximal end 120 thereof. Outer housing 116 includes a compartment 244 formed therein configured to house the wheel member 242a. Wheel member 242a includes an advancement wheel 246 mounted on an axle 248 that maintains the advancement wheel 246 within compartment 244 and allows the advancement wheel 246 to rotate in a first direction 208 and a second direction 210. An upper portion 246a of the advancement wheel 246 extends upwardly from compartment 244 and outside outer housing 116, so as to be engageable by an operator, while a bottom portion 246b of the advancement wheel 246 is positioned so as to be engaged with the inner housing 118, as explained in greater detail below.

As shown in FIG. 14, the advancement wheel 246 includes teeth 249 thereon that are configured to engage with a grooved track 250 formed along a top surface of the inner housing 118. With engagement of the teeth 249 and grooved track 250, the advancement wheel 246 and inner housing 118 thus form a rack and pinion type engagement that functions to urge the inner housing 118 (and fluid conduit 150) toward the distal end 122 of the outer housing 116 or back toward the proximal end 120 of the outer housing 116, depending on which direction the advancement wheel 246 is rotated.

In operation of the instrument delivery device 240, rotation of the advancement wheel 246 in a second direction 210 (i.e., toward the proximal end 120 of the outer housing 116) causes the rack and pinion type engagement of advancement wheel 246 and inner housing 118 to advance the inner housing 118 distally forward. The inner housing 118 thus moves/slides toward the distal end 122 of the outer housing 116, and this, in turn, causes the fluid conduit 150 to advance distally, such that the distal end 154 of the fluid conduit 150 may be extended out from outer housing 116 and blunted cannula 132 and into catheter assembly 10. Conversely, rotation of the wheel member 242 in a first direction 208 (i.e., toward the distal end 122 of the outer housing 116), causes the rack and pinion type engagement of advancement wheel 246 and inner housing 118 to retract the inner housing 118 proximally backward. The inner housing 118 thus moves/slides toward the proximal end 120 of the outer housing 116, and this, in turn, causes the fluid conduit 150 to retract proximally, such that the distal end 154 of the fluid conduit 150 may be retracted back into outer housing 116.

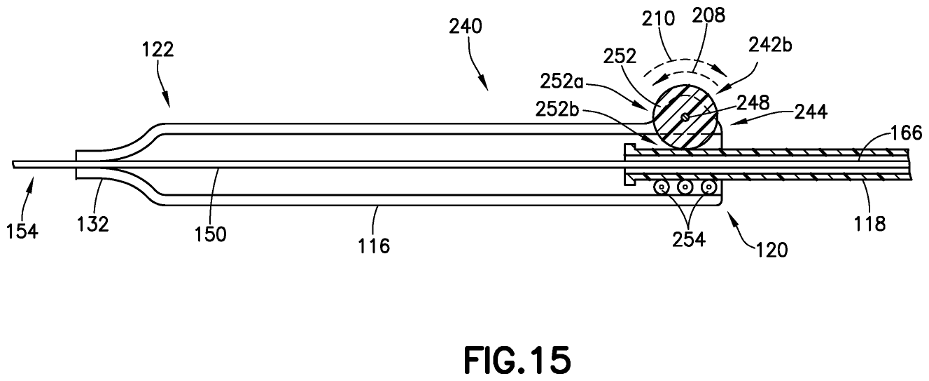

FIG. 15 illustrates another example embodiment of a wheel member 242—i.e., wheel member 242b—that may be included in instrument delivery device 240. The wheel member 242b is positioned on outer housing 116 at the proximal end 120 thereof. Wheel member 242b includes an advancement wheel 252 mounted on an axle 248 that maintains the advancement wheel 252 within a compartment 244 and allows the advancement wheel 252 to rotate in a first direction 208 and a second direction 210. An upper portion 252a of the advancement wheel 252 extends upwardly from compartment 244 and outside outer housing 116, so as to be engageable by an operator, while a bottom portion 252b of the advancement wheel 246 is positioned so as to engage and apply a pressure against the inner housing 118, as explained in greater detail below.

According to the illustrated embodiment, the wheel member 242b of instrument delivery device 240 is configured to interact with the inner housing 118 according to a friction-type engagement, in order to urge the inner housing 118 toward the distal end 122 of the outer housing 116 or back toward the proximal end 120 of the outer housing 116. To provide for a friction-type engagement between the wheel member 242b and the inner housing 118, the advancement wheel 252 may be formed of a rubber or other compliant material that assists in applying a pressure against a top surface of the inner housing 118. Additionally, one or more support wheels 254 are included in instrument delivery device 240 that are positioned opposite from the advancement wheel 252, i.e., adjacent a bottom surface of inner housing 118, and secured within the outer housing 116. Vertical alignment of the support wheels 254 and advancement wheel 252 on opposing sides of the inner housing 118, with a desired spacing therebetween that is slightly smaller than a diameter/height of the inner housing 118, creates a pressing force that is applied orthogonally against the inner housing 118. A friction engagement is thus provided between the advancement wheel 252 and the top surface of the inner housing 118 by which the inner housing 118 can be moved.

In operation of the instrument delivery device 240, rotation of the advancement wheel 252 in a second direction 210 (i.e., toward the proximal end 120 of the outer housing 116) causes the friction engagement between the advancement wheel 252 and inner housing 118 to advance the inner housing 118 distally forward. The inner housing 118 thus moves/slides toward the distal end 122 of the outer housing 116, and this, in turn, causes the fluid conduit 150 to advance distally, such that the distal end 154 of the fluid conduit 150 may be extended out from outer housing 116 and blunted cannula 132 and into catheter assembly 10. Conversely, rotation of the advancement wheel 252 in a first direction 208 (i.e., toward the distal end 122 of the outer housing 116), causes the friction engagement between the advancement wheel 252 and inner housing 118 to retract the inner housing 118 proximally backward. The inner housing 118 thus moves/slides toward the proximal end 120 of the outer housing 116, and this, in turn, causes the fluid conduit 150 to retract proximally, such that the distal end 154 of the fluid conduit 150 may be retracted back into outer housing 116.

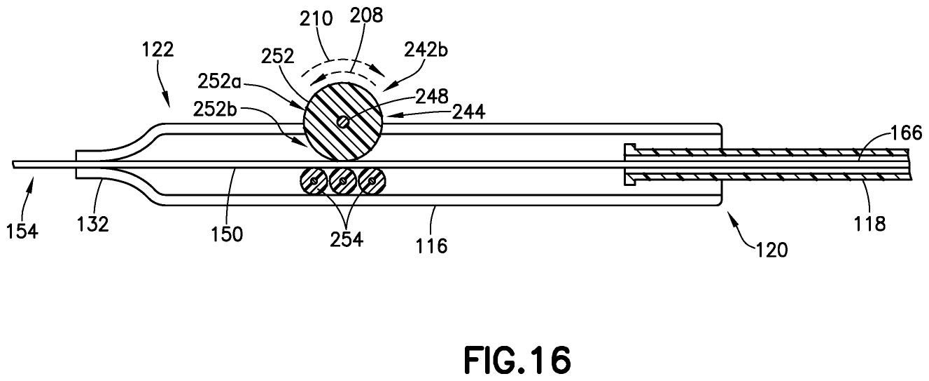

In another embodiment, the wheel member 242b and support wheel(s) 254 of FIG. 15 may be repositioned on/in outer housing 116 toward the distal end 122 thereof and operate with one another to directly translate fluid conduit 150, as shown in FIG. 16. That is, the advancement wheel 252 is positioned such that an upper portion 252a of the advancement wheel 252 extends upwardly from compartment 244 and outside outer housing 116 and a bottom portion 252b of the advancement wheel 246 is positioned so as to engage the fluid conduit 150, while the support wheel(s) 254 are positioned opposite from the advancement wheel 252 and secured within the outer housing 116. The vertical alignment of the support wheels 254 and advancement wheel 252 on opposing sides of the fluid conduit 150, with a desired spacing therebetween that is slightly smaller than a diameter/height of the fluid conduit 150, creates a pressing force that is applied orthogonally against the fluid conduit 150. A friction engagement is thus provided between the advancement wheel 252 and the top surface of the fluid conduit 150 by which the fluid conduit 150 can be moved. In operation of the instrument delivery device 240, rotation of the advancement wheel 252 in a second direction 210 (i.e., toward the proximal end 120 of the outer housing 116) causes the friction engagement between the advancement wheel 252 and fluid conduit 150 to advance the fluid conduit 150 (and thus also inner housing 118) distally forward. Conversely, rotation of the advancement wheel 252 in a first direction 208 (i.e., toward the distal end 122 of the outer housing 116), causes the friction engagement between the advancement wheel 252 and fluid conduit 150 to retract the fluid conduit 150 (and thus also inner housing 118) proximally backward.

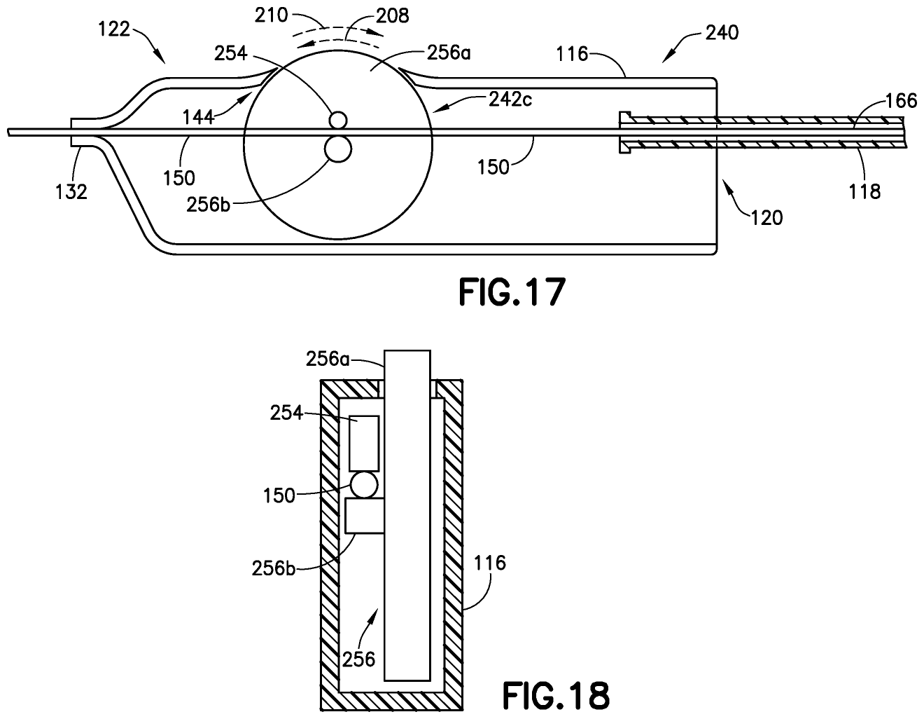

As described above regarding the embodiments of FIGS. 12-16, rotation of the advancement wheel 246, 252 in a second direction 210 (i.e., toward the proximal end 120 of the outer housing 116) causes the inner housing 118 to advance distally forward, while rotation of the advancement wheel 246, 252 in a first direction 208 (i.e., toward the distal end 122 of the outer housing 116), causes the inner housing 118 to retract proximally backward. According to additional embodiments, it is recognized that the structure of the advancement wheel 246, 252 and the positioning of the inner housing 118 (within outer housing 116) could be modified to reverse the direction of movement of the inner housing 118 relative to the direction of rotation of the advancement wheel 246, 252. Referring to FIGS. 17 and 18, a wheel member 242c is provided on instrument delivery device 240 that includes a two-tiered advancement wheel 256—with advancement wheel 256 including an actuation portion 256a and an engagement portion 256b. The portions 256a, 256b are integrally formed, with the actuation portion 256a having a larger diameter than the engagement portion 256b. The actuation portion 256a extends upwardly from compartment 244 and outside outer housing 116, so as to be engageable by an operator, while the engagement portion 256b is positioned so as to engage and apply a pressure against the fluid conduit 150, as explained in greater detail below. As shown best in FIG. 17, the inner housing 118 and fluid conduit 150 are positioned within outer housing 116 and relative to wheel member 242c and to and support wheel 254 such that a bottom surface of fluid conduit 150 rides on top of the engagement portion 256b and a top surface on fluid conduit 150 is engaged by support wheel 254. This construction of wheel member 242c and arrangement of support wheel 254 relative 254, along with positioning of fluid conduit 150, results in rotation of the advancement wheel 256 in the first direction 208 (i.e., toward the distal end 122 of the outer housing 116) causing the fluid conduit 150 to advance distally forward and rotation of the advancement wheel 256 in the second direction 210 (i.e., toward the proximal end 120 of the outer housing 116) causing the fluid conduit 150 to retract proximally backward.

It is recognized that a similar type of construction of wheel member 242c could be positioned at the proximal end 120 of outer housing 116 rather than the position shown in FIG. 17, with the inner housing 118 being engaged by engagement portion 256b and support wheel 254 instead of fluid conduit 150. Still further, it is recognized that, with the wheel member 242c positioned at the proximal end 120 of outer housing 116, engagement portion 256b of advancement wheel 256 may be structured to engage the inner housing 118 via a rack and pinion type engagement, as previously described in the embodiment of FIG. 14, rather than a friction type engagement.

Beneficially, embodiments of the instrument delivery devices described herein provide an ergonomic, one-handed advancement option for an operator. Positioning of a wheel member on the distal outer housing of the instrument delivery device enables the operator to use a single hand to both steady the instrument delivery device relative to a catheter assembly 10 and operate the wheel member to advance and/or retract an instrument. This allows the advancement hand of the operator to stay in a reference position without requiring significant motion axially following the motion of the inner housing, therefore simplifying use of the instrument delivery device.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. An instrument delivery device for advancing an instrument into a vascular access device, the instrument delivery device comprising:
an outer housing defining an inner volume and having a proximal end and a distal end;
an introducer portion positioned at the distal end of the outer housing and configured to mate with an access connector of the vascular access device;
an inner housing having a proximal end and a distal end, with the distal end positioned within the inner volume and engaged with a proximal end of the instrument, and with the inner housing configured to move relative to the outer housing; and
a wheel member positioned on the outer housing and rotatable relative to the outer housing, wherein the wheel member is operably coupled to the inner housing to move the inner housing distally within the outer housing when the wheel member is rotated;
wherein the distal movement of the inner housing moves the instrument from a first position, in which a distal end of the instrument is disposed within the outer housing, to a second position, in which the distal end of the instrument is disposed beyond the distal end of the outer housing.

2. The instrument delivery device of claim 1, wherein the wheel member is mounted on the outer housing adjacent the distal end of the outer housing.

3. The instrument delivery device of claim 2, further comprising a grip feature formed on the outer housing, at the distal end of the outer housing adjacent to the wheel member.

4. The instrument delivery device of claim 2, wherein the wheel member comprises:
an advancement wheel engageable by the user to cause rotation thereof; and
a spool operably coupled with the advancement wheel such that the spool rotates responsive to rotation of the advancement wheel.

5. The instrument delivery device of claim 4, further comprising a tether cord having a first end coupled to the distal end of the inner housing and a second end engaged with the spool.

6. The instrument delivery device of claim 4, wherein rotation of the wheel member in a first direction causes the tether cord to wrap around the spool and pull the inner housing toward the distal end of the outer housing, thereby moving the instrument from the first position to the second position.

7. The instrument delivery device of claim 5, wherein the tether cord comprises a semi-rigid cord, and wherein rotation of the wheel member in a second direction causes the semi-rigid cord to unwrap from the spool and push the inner housing back toward the proximal end of the outer housing, thereby moving the instrument from the second position to the first position.

8. The instrument delivery device of claim 4, wherein the spool is mounted adjacent the advancement member and includes a gear mounted thereto, with each of the advancement member and the gear including teeth thereon that mate together such that rotation of the advancement member causes a rotation of the gear that is transferred to the spool.

9. The instrument delivery device of claim 8, wherein the advancement member and the gear have different diameters, so as to provide a mechanical advantage of force or displacement when pulling the tether cord as the advancement member is rotated.

10. The instrument delivery device of claim 4, wherein the instrument runs forward from the proximal end to the distal end of the outer housing, and wherein one of the instrument and the tether cord is looped around the spool one or more times to effectively couple the instrument or the tether cord to the wheel member.

11. The instrument delivery device of claim 10, wherein rotation of the wheel member in a first direction causes the instrument and the inner housing to advance toward the distal end of the outer housing, thereby moving the instrument from the first position to the second position; and
wherein rotation of the wheel member in a second direction causes the instrument and the inner housing to retract toward the proximal end of the outer housing, thereby moving the instrument from the second position to the first position.

12. The instrument delivery device of claim 1, wherein during rotation of the wheel member in the first direction and the second direction, a first portion of the instrument is pulled by the wheel member and a second portion of the instrument is pushed by the wheel member.

13. The instrument delivery device of claim 1, wherein the wheel member is mounted on the outer housing adjacent the proximal end of the outer housing, with a bottom side of the wheel member engaging an upper surface of the inner housing; and
wherein rotation of the wheel member in a second direction, toward the proximal end of the outer housing, causes the bottom side of the wheel member to urge the inner housing toward the distal end of the outer housing, thereby moving the instrument from the first position to the second position.

14. The instrument delivery device of claim 13, wherein the wheel member comprises a toothed wheel and the top surface of the inner housing comprises a grooved track, with the toothed wheel engaging the grooved track in a rack and pinion type engagement as the toothed wheel is rotated in the second direction, to urge the inner housing toward the distal end of the outer housing.

15. The instrument delivery device of claim 13, further comprising one or more support wheels positioned in the outer housing, within the inner volume, and adjacent a bottom surface of the inner housing, opposite the wheel member, with the wheel member and the one or more support wheels pressing together on the inner housing to provide a friction engagement between the wheel member and the top surface of the inner housing; and
wherein the friction engagement between the wheel member and the top surface of the inner housing urges the inner housing toward the distal end of the outer housing as the wheel member is rotated in the second direction.

16. The instrument delivery device of claim 15, wherein the wheel member comprises a compliant advancement wheel configured to provide the friction engagement between the wheel member and the top surface of the inner housing.

17. The instrument delivery device of claim 1, wherein the instrument is one or more of a catheter, guidewire, obturator, wire, electrical wiring, probe, light pipe, and sensor.

18. The instrument delivery device of claim 1, wherein the instrument is a catheter, and wherein the inner housing is in fluid communication with the catheter, such that fluid flowing proximally from the catheter is received within the inner housing or fluid flowing distally from the inner housing is received in the catheter.

19. The instrument delivery device of claim 1, further comprising one or more supports arranged within the outer housing and configured to limit buckling of the instrument as it is advanced through the outer housing.

20. A system, comprising:
a catheter assembly comprising:
a catheter adapter, including:
a lumen extending between a distal end and a proximal end of the catheter adapter; and
a side port arranged between the distal end and the proximal end, the side port in fluid communication with the lumen;
a catheter secured to the distal end of the catheter adapter and extending distally from the catheter adapter; and
a fluid conduit having a proximal end coupled to the side port and a distal end coupled to a needless access connector; and
the instrument delivery device of claim 1, wherein the instrument delivery device advances an instrument through the needless access connector, the fluid conduit, and the catheter adapter, and into the catheter.
